(19) United States
(12) Patent Application Publication
(10) Pub. No.: US 2013/0160767 A1
(43) Pub. Date: Jun. 27, 2013

(54) EXERCISE MACHINE INCLUDING OXYGEN DISPENSER

(71) Applicant: Vera Abella, Key West, FL (US)

(72) Inventor: Vera Abella, Key West, FL (US)

(21) Appl. No.: 13/727,129

(22) Filed: Dec. 26, 2012

Related U.S. Application Data
(60) Provisional application No. 61/580,266, filed on Dec. 26, 2011.

(51) Int. Cl. A61M 16/10

(52) U.S. Cl. CPC A61M 16/10
USPC 128/204.22; 128/204.21

(57) ABSTRACT

An apparatus for providing oxygen to an individual engaging in exercise is disclosed. The apparatus includes a fastener for fastening the apparatus to an exercise machine, a container for holding a gas, a pump coupled with the container for pumping the gas from the container to a conduit, an arch element comprising an arch shaped structural element and a tubular element for receiving gas from the conduit, wherein the arch is disposed over the exercise machine, nozzles disposed in the arch element, wherein the nozzles receive gas from the tubular element and inject gas into the area where the individual resides on the exercise machine, and a processor communicatively coupled to the nozzles, wherein the processor is configured for transmitting control signals to the nozzles, and wherein the control signals are configured to command the nozzles to inject predefined amounts of gas.

### CROSS-REFERENCE TO RELATED APPLICATIONS

This patent application claims priority to provisional patent application 61/580,266 filed Dec. 26, 2011 and entitled “Exercise Machine Including Oxygen Dispenser.” The content of provisional patent application 61/580,266 is hereby incorporated by reference in its entirety.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### FIELD OF THE INVENTION

The invention disclosed broadly relates to the field of exercise equipment and accessories.

### BACKGROUND OF THE INVENTION

Exercising is an important part of a modern healthy lifestyle. Exercise is known to facilitate the maintenance of weight, burning of calories and release of stress. During exercise, an individual's blood flow and heart rate increases, thereby leading to an increased breathing rate. Consequently, an individual's need for oxygen increases during exercise, especially during strenuous exercise. One of the benefits of exercising outdoors is an increase in oxygen intake, which helps effective burning of body fat, lessens the effects of aging and aids the individual in producing more energy. Most of the U.S. population, however, lives in urban areas, where it is more difficult to perform outdoor workouts, and even when possible, the amount of oxygen available may be low, due to pollution and other factors. Thus, many individuals are left with no other choice but to work out at an indoor gym. Unfortunately, the air quality of the environment in an indoor gym may sometimes impede the body's ability to take in and absorb oxygen during exercise.

One cause of low air quality is air pollution. Air pollution is the introduction of chemicals, particulate matter, or biological materials that cause harm or discomfort to humans or other living organisms, or cause damage to the natural environment or built environment, into the atmosphere. Indoor air quality may also be affected by pollutants, which poses an obstacle to exercising indoors. Radon (Rn) gas, a carcinogen, is exuded from the Earth in certain locations and trapped inside houses. Building materials including carpeting and plywood emit formaldehyde (H2CO) gas. Paint and solvents give off volatile organic compounds (VOCs) as they dry. Lead paint can degenerate into dust and be inhaled. Intentional air pollution is introduced with the use of air fresheners, incense, and other scented items. Controlled wood fires in stoves and fireplaces can add significant amounts of smoke particulates into the air, inside and out. Pesticides and other chemical sprays used indoors without proper ventilation are additional causes of indoor air quality. Finally, indoor pollutants can include body odor and other body particulates, particularly in a gymnasium environment. Low indoor air quality can be annoying and uncomfortable for individuals, especially those exercising indoors.

Consequently, a need exists to overcome the problems with the prior art as discussed above, and particularly for a more efficient way of increasing indoor air quality and facilitating oxygen absorption during indoor exercise.

### SUMMARY OF THE INVENTION

Briefly, according to an embodiment of the present invention, an apparatus for providing oxygen to an individual engaging in exercise is disclosed. The apparatus includes a fastener for fastening the apparatus to an exercise machine, a container for holding a gas comprising oxygen or an oxygen mixture, a pump coupled with the container for pumping the gas from the container to a conduit, an arch element comprising an arch shaped structural element and a tubular element for receiving gas from the conduit, wherein the arch is disposed over the exercise machine substantially in an area wherein the individual resides on the exercise machine, one or more regulating nozzles disposed in the arch element and coupled to the tubular element, wherein the one or more regulating nozzles receive gas from the tubular element and wherein the one or more regulating nozzles inject gas into the area where the individual resides on the exercise machine, and a processor communicatively coupled to the one or more regulating nozzles, wherein the processor is configured for transmitting control signals to the one or more regulating nozzles, and wherein the control signals are configured to command the one or more regulating nozzles to inject predefined amounts of gas.

The foregoing and other features and advantages of the present invention will be apparent from the following more particular description of the preferred embodiments of the invention, as illustrated in the accompanying drawings.

### BRIEF DESCRIPTION OF THE DRAWINGS

The subject matter, which is regarded as the invention, is particularly pointed out and distinctly claimed in the claims at the conclusion of the specification. The foregoing and other features and also the advantages of the invention will be apparent from the following detailed description taken in conjunction with the accompanying drawings.

FIG. 1 is a block diagram showing the main components of an apparatus for providing oxygen to an individual engaging in exercise, according to one embodiment of the present invention.

FIG. 2A and FIG. 2B are illustrations of the apparatus of FIG. 1 shown in conjunction with an exercise machine, according to one embodiment of the present invention.

FIG. 2C is a block diagram showing the apparatus of FIG. 1 shown in conjunction with an exercise machine, according to one embodiment of the present invention.

FIG. 3 is a flowchart showing the method performed by the apparatus for providing oxygen to an individual engaging in exercise, according to one embodiment of the present invention.

FIG. 4 is a block diagram of a system including an example computing device and other computing devices.

### DETAILED DESCRIPTION

The present invention improves over the prior art by providing an apparatus that dispenses oxygen or an oxygen mixture directly to an individual while exercising indoors on an exercise machine, such as a treadmill, stationary bicycle, climbing machine or elliptical trainer. This apparatus thereby increases oxygen intake and absorption for an individual during indoor exercise, consequently offsetting low indoor air quality. The apparatus of the present invention is small, portable, has a minimum of moving parts and is easily attached to an exercise machine. The apparatus is further easily transferred from one exercise machine to another.

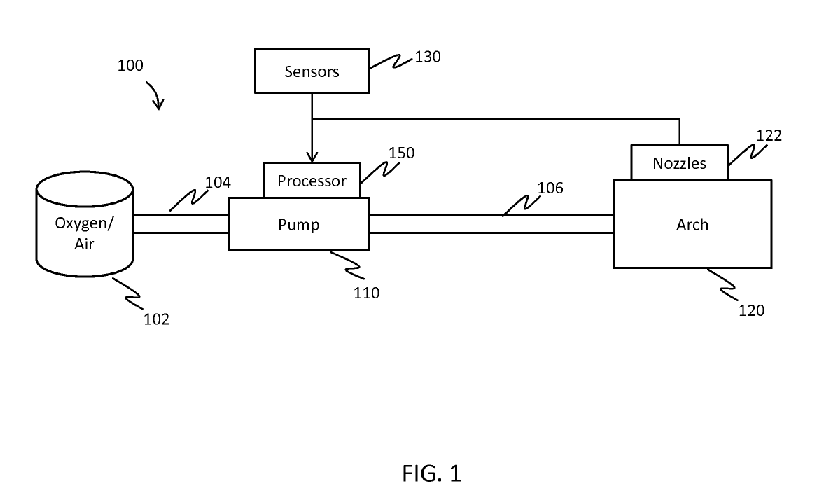

FIG. 1 is a block diagram showing the main components of an apparatus 100 for providing oxygen to an individual engaging in exercise, according to one embodiment of the present invention. FIG. 1 shows that the apparatus 100 includes a gas container 102, which contains a gas such as air, oxygen, or some combination of the two. Container 102 may also contain a scent or other olfactory element. The apparatus 100 further includes a pump 100 for pumping the gas from container 102 via conduit 104 to the arch element 120 via conduit 106. The gas pumped to the arch element 120 egresses via one more regulating nozzles 122 embedded in the arch element 120. A regulating nozzle comprises an aperture for egress of gas and a motor for opening and closing the aperture. The processor or computer 150 reads data from the sensor(s) 130 and controls the pump 110 and the nozzles 122.

In one embodiment, the sensor(s) 130 may comprise at least one of a temperature sensor, a humidity sensor, a mass flow sensor, a pressure sensor and a gas composition sensor. A temperature sensor provides temperature data while a pressure sensor provides pressure data. A humidity sensor measures the moisture content of a gas. A gas composition sensor may sense and report the pure substances in a gas. The gas composition sensor may also state for each substance its proportion of the gas mixture's molecule count. In one example, the gas composition sensor can measure the oxygen value of the ambient air, which is a relevant data value because the amount of oxygen in the ambient air affects the individual performing exercise. Thus, oxygen value of ambient air may be used to calibrate the amount of gas the apparatus 100 injects via arch 120. A humidity sensor may measure the moisture content of the ambient air, which may also affect the physiology of the individual performing exercise.

Note that regulating nozzles 122 are communicatively coupled with, and are controlled by, computer 150. Recall that a regulating nozzle comprises an aperture for egress of gas, and a motor for opening and closing the aperture. The motor of each regulating nozzle 122 reacts to commands received by computer 150, thereby affecting the amount of gas injected by said nozzles. FIG. 1 also shows that sensors 130 are communicatively coupled with, and transmit sensor data to, computer 150.

A prominent element of FIG. 1 is the processor or computer 150 associated, which may include a repository or database. Computer 150 is a central controller or operator for pump 110 and regulating nozzles 122. Computer 150 reads sensor data, calculates the amount of gas that shall be injected by each of the regulating nozzles 122, respectively, and then transmits control signals to the nozzles, wherein the control signals are configured to command the nozzles 122 to inject predefined amounts of gas for consumption by an individual using an exercise machine. The repository may serve data from a database, which is a repository for data used by computer 150 during the course of operation of the invention. The database may include one or more stored values representing an amount of gas to inject, wherein the stored values correspond to sensor data.

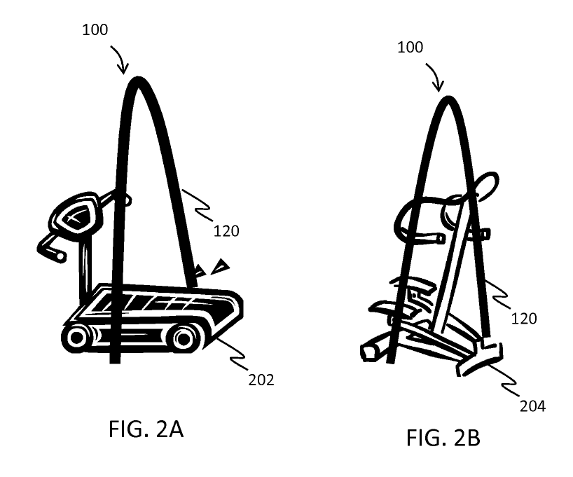

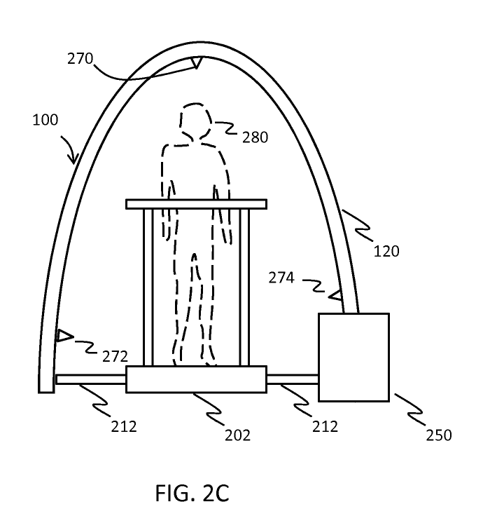

In one embodiment, the stored values are embedded in one or more lookup tables. The lookup table may comprise a data structure comprising a list or chart wherein each line or row lists data values or ranges of data values for sensor data. The data values or ranges of data values in the lookup table correspond to sensor data read in step 302 below. In one example, each line or row of the lookup table also includes a desired amount of gas that corresponds to the data values, or ranges, in that line or row. That is, the lookup table lists the desired amount of gas that should be injected by a particular nozzle, in order to provide optimal efficiency, for certain sensor data values or ranges of sensor data values. Therefore, each line or row of the lookup table may be seen as an if-then statement wherein the if-portion of the statement corresponds to sensor data values or ranges of sensor data values and the then-portion of the statement corresponds to a desired amount of gas that should be injected by a particular nozzle. In one embodiment, each nozzle 270, 272, 274 (see FIG. 2) may be associated with one or more lookup tables—that is, each nozzle may have one or more lookup tables that correspond to that specific nozzle.

FIG. 2A and FIG. 2B are illustrations of the apparatus 100 of FIG. 1 shown in conjunction with an exercise machine, according to one embodiment of the present invention. FIG. 2A shows apparatus 100 being used in conjunction with a treadmill 202. The present invention also anticipates the use of the apparatus 100 in conjunction with an elliptical training device including foot pads for an individual's feet, a stationary bicycle including a seat for an individual, a stepping exercise device including foot pads for an individual's feet, a rowing exercise machine including a seat for an individual, and a climbing exercise machine including foot pads for an individual's feet. FIG. 2B shows apparatus 100 being used in conjunction with an elliptical training device 204.

Notice that both FIG. 2A and FIG. 2B show the arch element 120 disposed over an exercise machine. An arch element 120 comprises an arch shaped structural element and a tubular element for receiving gas from the conduit 106, wherein the arch element is disposed over the exercise machine substantially in an area wherein the individual 280 resides on the exercise machine. This allows the gas emitted from the arch 120 to be emitted in an area where the individual 280 may consume it. The arch shaped structural element may be a structural element that has the ability to hold up the weight and structure of the portion of apparatus 100 that is hold over the exercise machine. The tubular element may be a pipe or other tube-like structure that transfers the gas from the conduit 106 to the nozzles 122.

FIG. 2C is a block diagram showing the apparatus 100 of FIG. 1 shown in conjunction with an exercise machine 202, according to one embodiment of the present invention. FIG. 2C shows an individual 280 using a treadmill 202. The arch element 120 is disposed over the exercise machine 202 and includes several regulating nozzles 270, 272, 274 disposed in the arch element 120 and coupled to the tubular element, wherein the regulating nozzles receive gas from the tubular element and inject gas into the area where the individual 280 resides on the exercise machine 202. FIG. 2C also shows a housing 250 which houses the various components of apparatus 100 such as the container 102, pump 110, processor 150 and conduit 104. Lastly, FIG. 2C shows one or more fasteners 212 that connect or couple the apparatus 100 to the exercise machine 202. In one embodiment, the fasteners 212 comprise a pair of fasteners, including one fastener on each side of the arch located at the bottom end of each end of the arch, wherein each fastener is coupled or connected to one side of the exercise machine 202.

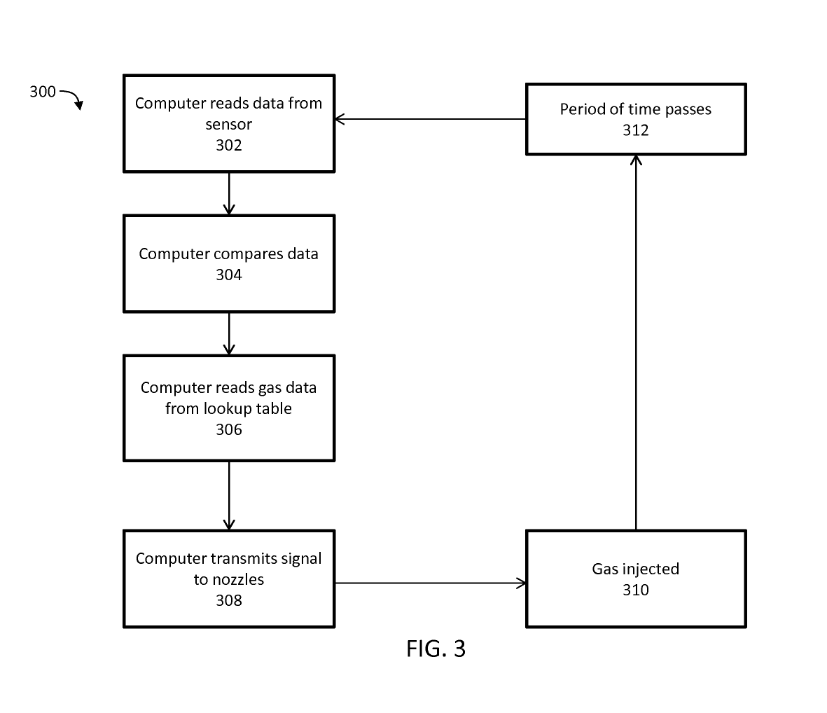

FIG. 3 is a flowchart showing the method 300 performed by the apparatus 100 for providing oxygen to an individual engaging in exercise, according to one embodiment of the present invention. Specifically, the method 300 describes how computer 150 reads data from the sensor 130, calculates the appropriate amounts of gas to inject and commands the nozzles 122 to inject said appropriate amounts of gas. Method 300 is described with reference to FIGS. 1 and 2A-2C above.

In a first step 302, the computer 150 reads sensor data in real time, or near real time, from the sensor 130. Sensor data from a temperature sensor may comprise a temperature value (such as in Celsius units) while sensor data from a pressure sensor may comprise a pressure value (such as in psi units) and sensor data from a humidity sensor may comprise a moisture content value (such as a percentage). Sensor data from a mass flow sensor may comprise a mass flow value (such as grams per second or density per second, i.e., grams per centimeter cubed per second). Sensor data from a gas composition sensor may comprise a gas composition value (such as ppm or percentage of volume or density, i.e., grams per centimeter cubed).

In step 304, the computer 150 compares a subset of the sensor data read in step 302 to data in a stored lookup table. Recall the lookup table lists the desired amount of gas that should be injected by a particular nozzle for certain sensor data values or ranges of sensor data values. Therefore, each line or row of the lookup table may be seen as an if-then statement wherein the if-portion of the statement corresponds to sensor data values or ranges of sensor data values and the then-portion of the statement corresponds to a desired amount of gas that should be injected by a particular nozzle. In one embodiment, the stored lookup table may be stored in volatile memory, such as RAM, or non-volatile memory, such as ROM, EPROM or flash memory. In step 304, the computer 150 finds a row in the lookup table that matches the sensor data read in step 302.

In step 306, the computer 150 reads from the lookup table the desired amount of gas corresponding to the matching line or row of the lookup table, which was identified in step 304. Note that in one embodiment, a single lookup table is used to define an amount of gas, if any, to be injected by the group of nozzles 122, respectively. In a second embodiment, a separate lookup table is used to define an amount of gas, if any, to be injected by each separate nozzle 270, 272, 274, respectively. In this second embodiment, steps 304, 306 are executed separately for each nozzle 270, 272, 274.

In step 308, the computer 150 transmits a control signal to one or more regulating nozzles 122, wherein each control signal is configured to command the respective regulating nozzle to inject the desired amount of gas that was read in step 306. In step 310, responsive to the signal received in step 308, the one or more regulating nozzles 122 respectively inject the amount of gas commanded by computer 150. In step 312, a set period of time passes. In one embodiment, step 312 includes the passing of 500 milliseconds. Subsequently, control flows immediately back to step 302 wherein steps 302 through 312 are executed periodically.

Note that the cyclical process of method 300 involves the computer 150 using feedback data from the sensors to confirm the appropriate amount of gas to inject at various times. This feedback loop is performed periodically, such as every 500 milliseconds, so as to ensure optimal functioning and provide quick reactions to changing conditions.

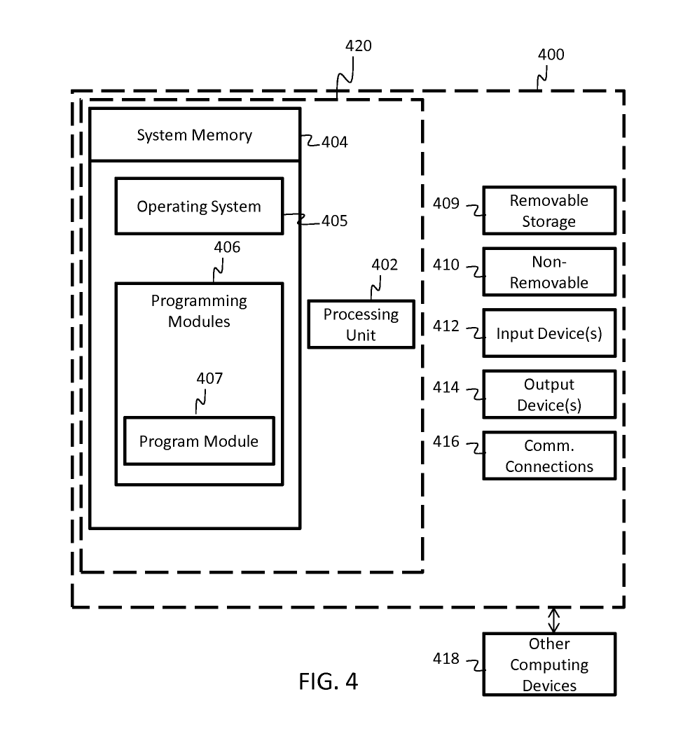

FIG. 4 is a block diagram of a system including an example computing device 400 and other computing devices. Consistent with the embodiments described herein, the aforementioned actions performed by processor 150 may be implemented in a computing device, such as the computing device 400 of FIG. 4. Any suitable combination of hardware, software, or firmware may be used to implement the computing device 400. The aforementioned system, device, and processors are examples and other systems, devices, and processors may comprise the aforementioned computing device. Furthermore, computing device 400 may comprise an operating environment for the method 300 above.

With reference to FIG. 4, a system consistent with an embodiment of the invention may include a plurality of computing devices, such as computing device 400. In a basic configuration, computing device 400 may include at least one processing unit 402 and a system memory 404. Depending on the configuration and type of computing device, system memory 404 may comprise, but is not limited to, volatile (e.g. random access memory (RAM)), non-volatile (e.g. read-only memory (ROM)), flash memory, or any combination or memory. System memory 404 may include operating system 405, one or more programming modules 406 (such as program module 407). Operating system 405, for example, may be suitable for controlling computing device 400′s operation. In one embodiment, programming modules 406 may include, for example, a program module 407. Furthermore, embodiments of the invention may be practiced in conjunction with a graphics library, other operating systems, or any other application program and is not limited to any particular application or system. This basic configuration is illustrated in FIG. 4 by those components within a dashed line 420.

Computing device 400 may have additional features or functionality. For example, computing device 400 may also include additional data storage devices (removable and/or non-removable) such as, for example, magnetic disks, optical disks, or tape. Such additional storage is illustrated in FIG. 4 by a removable storage 409 and a non-removable storage 410. Computer storage media may include volatile and nonvolatile, removable and non-removable media implemented in any method or technology for storage of information, such as computer readable instructions, data structures, program modules, or other data. System memory 404, removable storage 409, and non-removable storage 410 are all computer storage media examples (i.e. memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 400. Any such computer storage media may be part of device 400. Computing device 400 may also have input device(s) 412 such as a keyboard, a mouse, a pen, a sound input device, a camera, a touch input device, etc. Output device(s) 414 such as a display, speakers, a printer, etc. may also be included. The aforementioned devices are only examples, and other devices may be added or substituted.

Computing device 400 may also contain a communication connection 416 that may allow device 400 to communicate with other computing devices 418, such as over a network in a distributed computing environment, for example, an intranet or the Internet. Communication connection 416 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both computer storage media and communication media.

As stated above, a number of program modules and data files may be stored in system memory 404, including operating system 405. While executing on processing unit 402, programming modules 406 may perform processes including, for example, one or more of the methods 300 above. The aforementioned processes are examples, and processing unit 402 may perform other processes. Other programming modules that may be used in accordance with embodiments of the present invention may include electronic mail and contacts applications, word processing applications, spreadsheet applications, database applications, slide presentation applications, drawing or computer-aided application programs, etc.

Generally, consistent with embodiments of the invention, program modules may include routines, programs, components, data structures, and other types of structures that may perform particular tasks or that may implement particular abstract data types. Moreover, embodiments of the invention may be practiced with other computer system configurations, including hand-held devices, multiprocessor systems, microprocessor-based or programmable consumer electronics, minicomputers, mainframe computers, and the like. Embodiments of the invention may also be practiced in distributed computing environments where tasks are performed by remote processing devices that are linked through a communications network. In a distributed computing environment, program modules may be located in both local and remote memory storage devices.

Furthermore, embodiments of the invention may be practiced in an electrical circuit comprising discrete electronic elements, packaged or integrated electronic chips containing logic gates, a circuit utilizing a microprocessor, or on a single chip (such as a System on Chip) containing electronic elements or microprocessors. Embodiments of the invention may also be practiced using other technologies capable of performing logical operations such as, for example, AND, OR, and NOT, including but not limited to mechanical, optical, fluidic, and quantum technologies. In addition, embodiments of the invention may be practiced within a general purpose computer or in any other circuits or systems.

Embodiments of the present invention, for example, are described above with reference to block diagrams and/or operational illustrations of methods, systems, and computer program products according to embodiments of the invention. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the invention have been described, other embodiments may exist. Furthermore, although embodiments of the present invention have been described as being associated with data stored in memory and other storage mediums, data can also be stored on or read from other types of computer-readable media, such as secondary storage devices, like hard disks, floppy disks, or a CD-ROM, or other forms of RAM or ROM. Further, the disclosed methods' stages may be modified in any manner, including by reordering stages and/or inserting or deleting stages, without departing from the invention.

Although specific embodiments of the invention have been disclosed, those having ordinary skill in the art will understand that changes can be made to the specific embodiments without departing from the spirit and scope of the invention. The scope of the invention is not to be restricted, therefore, to the specific embodiments. Furthermore, it is intended that the appended claims cover any and all such applications, modifications, and embodiments within the scope of the present invention.

What is claimed is:

1. An apparatus for providing oxygen to an individual engaging in exercise, comprising:
a fastener for fastening the apparatus to an exercise machine;
a container for holding a gas comprising oxygen or an oxygen mixture;
a pump coupled with the container for pumping the gas from the container to a conduit;
an arch element comprising an arch shaped structural element and a tubular element for receiving gas from the conduit, wherein the arch element is disposed over the exercise machine substantially in an area wherein the individual resides on the exercise machine;
one or more regulating nozzles disposed in the arch element and coupled to the tubular element, wherein the one or more regulating nozzles receive gas from the tubular element and wherein the one or more regulating nozzles inject gas into the area where the individual resides on the exercise machine; and
a processor communicatively coupled to the one or more regulating nozzles, wherein the processor is configured for transmitting control signals to the one or more regulating nozzles, and wherein the control signals are configured to command the one or more regulating nozzles to inject predefined amounts of gas.

2. The apparatus of claim 1, wherein a regulating nozzle comprises:
an aperture for egress of gas, and a motor for opening and closing the aperture.

3. The apparatus of claim 2, further comprising:
a sensor comprising at least one of a temperature sensor, a humidity sensor, a mass flow sensor, a pressure sensor and a gas composition sensor, and wherein the sensor is communicatively coupled with the processor.

4. The apparatus of claim 3, wherein the processor is further configured for:
reading sensor data from the sensor;
calculating an amount of gas to be injected by the one or more regulating nozzles, respectively, based on the sensor data; and
transmitting control signals to the one or more regulating nozzles, wherein the control signals are configured to command the one or more regulating nozzles to inject an amount of gas, respectively.

5. The apparatus of claim 4, wherein the exercise machine comprises an elliptical training device including foot pads for an individual's feet.

6. The apparatus of claim 4, wherein the exercise machine comprises a stationary bicycle including a seat for an individual.

7. The apparatus of claim 4, wherein the exercise machine comprises a stepping exercise device including foot pads for an individual's feet;

8. The apparatus of claim 4, wherein the exercise machine comprises a rowing exercise machine including a seat for an individual.

9. The apparatus of claim 4, wherein the exercise machine comprises a climbing exercise machine including foot pads for an individual's feet.

10. An apparatus for providing oxygen to an individual engaging in exercise, comprising:
a fastener for fastening the apparatus to an exercise machine;
a container for holding a gas comprising oxygen or an oxygen mixture;
a pump coupled with the container for pumping the gas from the container to a conduit;
an arch element comprising an arch shaped structural element and a tubular element for receiving gas from the conduit, wherein the arch is disposed over the exercise machine substantially in an area wherein the individual resides on the exercise machine;
one or more regulating nozzles disposed in the arch element and coupled to the tubular element, wherein the one or more regulating nozzles receive gas from the tubular element and wherein the one or more regulating nozzles inject gas into the area where the individual resides on the exercise machine;
a sensor comprising at least one of a temperature sensor, a humidity sensor, a mass flow sensor, a pressure sensor and a gas composition sensor;
a processor communicatively coupled to the one or more regulating nozzles and the sensor, wherein the processor is configured for:
reading sensor data from the sensor;
calculating an amount of gas to be injected by the one or more regulating nozzles, respectively, based on the sensor data; and
transmitting control signals to the one or more regulating nozzles, wherein the control signals are configured to command the one or more regulating nozzles to inject predefined amounts of gas, respectively.

11. The apparatus of claim 10, wherein a regulating nozzle comprises:
an aperture for egress of gas, and a motor for opening and closing the aperture.

12. The apparatus of claim 11, wherein the exercise machine comprises an elliptical training device including foot pads for an individual's feet.

13. The apparatus of claim 11, wherein the exercise machine comprises a stationary bicycle including a seat for an individual.

14. The apparatus of claim 11, wherein the exercise machine comprises a stepping exercise device including foot pads for an individual's feet;

15. The apparatus of claim 11, wherein the exercise machine comprises a rowing exercise machine including a seat for an individual.

16. The apparatus of claim 11, wherein the exercise machine comprises a climbing exercise machine including foot pads for an individual's feet.
